(19) United States
(12) Patent Application Publication
(10) Pub. No.: US 2007/0093684 A1
(43) Pub. Date: Apr. 26, 2007

(54) Extra-aortic patch

(75) Inventor: William Suttle Peters, Auckland (NZ)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Sunshine Heart Company Pty Ltd.

(21) Appl. No.: 10/595,590

(22) PCT Filed: Oct. 28, 2004
(86) PCT No.: PCT/AU04/01483

§ 371 Date: Apr. 28, 2006

(30) Foreign Application Priority Data
Oct. 30, 2003 (AU) 2003905993

(51) Int. Cl. A61M 1/10

(52) U.S. Cl. 600/18

(57) ABSTRACT

A method of heart assistance including the step of directly attaching a heart assist device (12) including an inflatable balloon or chamber (18) to, the exterior of an arterial vessel (10).

### FIELD OF THE INVENTION

The present invention relates generally to a counter-pulsation heart assist device, system and method and, more particularly, to an extra-aortic patch and a heart assist device and method using aortic deformation.

### BACKGROUND OF THE INVENTION

The Applicant's International PCT Patent Application Nos. PCT/AU00/00654 and PCT/AU02/00974 disclose various counter-pulsation heart assist devices that utilise aortic deformation. The contents of these specifications are hereby incorporated herein by cross reference.

Known counter-pulsation heart assist devices generally include an inelastic shell with a flexible membrane sealingly attached to the periphery of the shell. The membrane defines an inflatable space between it and the interior of the shell. The shell also has an inlet/outlet port which is adapted for connection to a motive means that can periodically introduce, and withdraw, a fluid to and from the space in counter-pulsation with the patient's heart rhythm. A substantially inelastic, flexible wrap is placed around an arterial vessel and over the device to secure the device adjacent the exterior of the vessel. The balloon is normally positioned on the radially outer side of the ascending aorta.

It is the object of the present invention to provide an alternative means for securing a heart assist device adjacent an arterial vessel.

### SUMMARY OF THE INVENTION

Accordingly, in a first aspect, the present invention provides a method of heart assistance including the step of directly attaching a heart assist device including an inflatable balloon or chamber to the exterior of an arterial vessel.

In one embodiment, the balloon or chamber is itself attached to the arterial vessel.

In another embodiment, a shroud forming a part of the heart assist device and overlying the balloon or chamber is attached to the vessel to hold the balloon or chamber in contact with the vessel. The shroud or the balloon or chamber is preferably attached to the aorta around its circumferential periphery.

In one embodiment, the method includes the step of directly attaching the shroud of the heart assist device to an arterial vessel with the associated inflatable balloon or chamber secured beneath the shroud and adjacent the vessel.

The method preferably includes suturing the shroud to the vessel, most preferably with non-absorbable sutures, unless the device is intended to be removed remotely at a latter date. Alternatively, the shroud can be glued to the vessel. As a further alternative, the shroud can be stapled or clipped to the vessel.

The balloon or chamber is preferably attached at substantially all of its surface exterior that is disposed adjacent to the vessel exterior.

In another embodiment, the method includes the step of directly attaching the balloon or chamber of the heart assist device to an arterial vessel with an associated shroud or wrap secured over the balloon or chamber and onto the vessel.

The method preferably includes gluing the balloon or chamber to the vessel, most preferably with fibrin or another natural adhesive protein.

The method preferably includes the step of sequentially introducing and withdrawing a fluid into and from the balloon or chamber in counterpulsation with the arterial vessel so as to bring about the heart assistance.

In a second aspect, the present invention provides a heart assist device including a shroud or wrap and an inflatable balloon or chamber, wherein the shroud or wrap has a larger peripheral extent than that of the balloon or chamber, and at least some of the periphery of the shroud or wrap is adapted for direct attachment to the arterial vessel.

The shroud periphery is preferably suturable to the vessel. The sutures are preferably non-absorbable. If the balloon is positioned over the descending thoracic aorta, the shroud periphery is sutured to the intercostal fascia and fascia overlying the vertebral column.

In another form, the shroud periphery is adapted for gluing to the vessel.

As a further alternative, the shroud periphery is adapted for stapling or clipping to the vessel.

In a third aspect, the present invention provides a method of heart assistance, the method including the steps of gluing an inflatable balloon or chamber of a heart assist device to a wall of an arterial vessel and inflating the balloon or chamber to cause inward displacement of the wall in the region that is adjacent the balloon or chamber.

### BRIEF DESCRIPTION OF THE DRAWINGS

A preferred embodiment of the invention will now be described, by way of an example only, with reference to the accompanying drawings in which:

FIG. 1 is a schematic perspective view of an aorta of a patient with a first embodiment of a device according to the invention attached thereto;

FIG. 2 is a schematic cross section view of the aorta and device shown in FIG. 1 along line 2-2; and

FIG. 3 is a schematic cross section view of an aorta and a second embodiment of device according to the invention attached thereto.

### DETAILED DESCRIPTION OF THE PREFERRED EMBODIMENTS

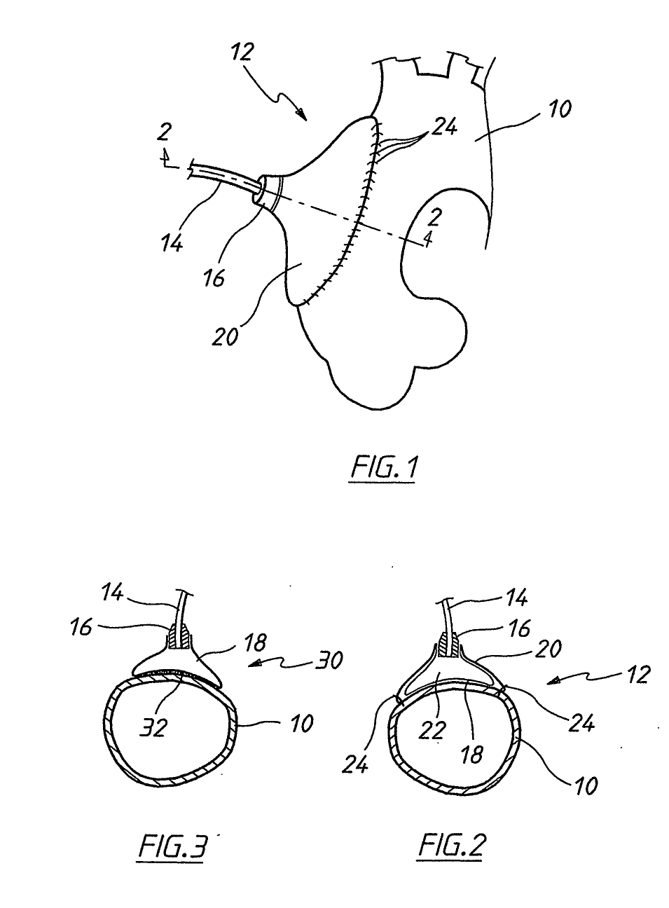

FIG. 1 is a schematic perspective view of an ascending aorta 10 and a heart assist device 12 according to a first embodiment of the invention. The device 12 has a fluid tube 14 for connection to a motive power source (not shown), which sealingly engages a bushing 16. A flexible balloon membrane 18 (see FIG. 2) is sealingly attached to the bushing 16. The balloon 18 is formed from a polyurethane, polyurethane-silicone co-polymer, silicone, or similar material

The balloon 18 is protected by an inelastic, shroud 20, which snugly engages the bushing 16 and sealingly sandwiches the open end of the balloon 18 therebetween. The shroud 20 has a larger peripheral extent (ie. is wider) than the balloon 18. The balloon 18 defines an inflatable space 22. The shroud 20 can be formed in part of whole of polyurethane, polyurethane-silicone co-polymer, silicone, polyester, or similar materials.

The device 12 is secured on the radially outer side of the ascending aorta 10 by the shroud 20 being directly attached to the aorta 10 by one or two rows of non absorbable sutures 24 along the sides of the shroud 20. The sutures 24 are preferably of the mono-filament type, such as Prolene 3/0 (Trade Mark), but may be any nonabsorbable material.

In operation, the motive means periodically introduces, and withdraws, a fluid (e.g. a gas such as helium or air or a liquid such as a saline solution or an oil) to and from the space 22 in counter-pulsation with the patient's heart rhythm. When fluid is introduced into the space 22, the balloon 18 expands and the aorta's external wall is compressed and inwardly deformed until it is close to but not abutting the aorta's opposite interior wall. When fluid is withdrawn from the space 22, the balloon retracts to the configuration shown in FIG. 2 and the aorta 10 returns to normal position allowing maximum blood flow therethrough.

FIG. 3 is a schematic cross-sectional view of an ascending aorta 10 and a heart assist device 30 according to a second embodiment of the invention. Like features to those of the first embodiment will be denoted with like reference numerals in relation to the second embodiment. The device 30 differs from the device 12 in that the balloon 18 is itself directly attached to the aorta 10 by glue at (darkened) region 32.

The advantages of the above devices include that they are relatively easier and safer to implant compared to known surgical procedures because they are not in the blood stream, and because the ascending aorta does not need to be completely mobilised free of the pulmonary artery. The second embodiment also allows for placement of aorto-coronary bypass grafts to the ascending aorta, separate from the device 30. Additionally, as the back part of the aorta is not attached to the device, the ascending aorta retains a majority of its anisotropic elastic nature which is important to minimise any loss of aortic compliance. Such devices may be particularly useful in patients having re-do surgery, where scar tissue may make complete mobilisation of the aorta from the pulmonary artery difficult. Additionally, redo patients may have patent aorto-coronary bypass grafts, that can be retained on the aorta. The devices described above also allows for growth and/or dilation of the aorta over time and as such may be suitable for use in younger patients where the aorta is smaller, more elastic and growing, or in patients who have been in severe heart failure and the ascending aorta is smaller than normal for any given age, due to chronically low cardiac output. The advantages of such a device and method on the descending aorta is that, due to presence of multiple side-branches, circumferential wrapping is not easily achieved, and “patch” attachment is more achievable. Further, with the descending thoracic aorta, a longer length is made available and thus a larger balloon can be used. Finally, the devices described above advantageously reduce the amount of foreign material introduced into a patient's body.

It would be appreciated by persons skilled in the art that numerous variations and/or modifications may be made to the invention as shown in the specific embodiment without departing from the spirit or scope of the invention as broadly described. For example, surgical glue could be used in place of the sutures.

1. A method of heart assistance including the step of directly attaching a heart assist device including an inflatable balloon or chamber to the exterior of an arterial vessel.

2. The method as claimed in claim 1, wherein the balloon or chamber is itself attached to the arterial vessel.

3. The method as claimed in claim 1, wherein a shroud forming a part of the heart assist device and overlying the balloon or chamber is attached to the vessel to hold the balloon or chamber in contact with the vessel.

4. The method as claimed in claim 1, wherein the shroud or the balloon or chamber is attached to the aorta around its circumferential periphery.

5. The method as claimed in claim 3, wherein the method includes the step of directly attaching the shroud of the heart assist device to an arterial vessel with the associated inflatable balloon or chamber secured beneath the shroud and adjacent the vessel.

6. The method as claimed in claim 5, wherein the method includes suturing the shroud to the vessel.

7. The method as claimed in claim 6, wherein the method includes suturing the shroud to the vessel with non-absorbable sutures.

8. The method as claimed in claim 5, wherein the method includes gluing the shroud to the vessel.

9. The method as claimed in claim 5, wherein the method includes stapling the shroud to the vessel.

10. The method as claimed in claim 5, wherein the method includes clipping the shroud to the vessel.

11. The method as claimed in claim 5, wherein the balloon or chamber is attached at substantially all of its surface exterior that is disposed adjacent to the vessel exterior.

12. The method as claimed in claim 1, wherein the method includes the step of directly attaching the balloon or chamber of the heart assist device to an arterial vessel with an associated shroud or wrap secured over the balloon or chamber and onto the vessel.

13. The method as claimed in claim 12, wherein the method includes gluing the balloon or chamber to the vessel.

14. The method as claimed in claim 1, wherein the method includes the step of directly attaching the balloon or chamber of the heart assist device to an arterial vessel with an associated fibrin or another natural adhesive protein secured over the balloon or chamber and onto the vessel.

15. The method as claimed in claim 1, wherein the method includes the step of sequentially introducing and withdrawing a fluid into and from the balloon or chamber in counterpulsation with the arterial vessel.

16. A heart assist device including a shroud or wrap and an inflatable balloon or chamber, wherein the shroud or wrap has a larger peripheral extent than that of the balloon or chamber, and at least some of the periphery of the shroud or wrap is adapted for direct attachment to the arterial vessel.

17. The device as claimed in claim 16, wherein the shroud periphery is adapted for suturing to the vessel.

18. The device as claimed in claim 17, wherein the shroud periphery is sutured to the intercostal fascia and fascia overlying the vertebral column.

19. The device as claimed in claim 16, wherein the shroud periphery is adapted for gluing to the vessel.

20. The method as claimed in claim 16, wherein the shroud periphery is adapted for stapling to the vessel.

21. The device as claimed in claim 16, wherein the shroud periphery is adapted for clipping to the vessel.

22. A method of heart assistance, the method including the steps of gluing an inflatable balloon or chamber of a heart assist device to a wall of an arterial vessel and inflating the balloon or chamber to cause inward displacement of the wall in the region that is adjacent the balloon or chamber.
